SEQUENCE LISTING (2 sequences)

SEQ ID NO: 1 - protein, 608 aa, synthetic construct
MYRNLIIATALTCGAYSAYVPSEPWSTLTPDASLESALKDYSQTFGIAIKSLDADKIKRDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 2 - protein, 546 aa, synthetic construct
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

(19) United States
(12) Patent Application Publication
(10) Pub. No.: US 2026/0295109 A1
(43) Pub. Date: Oct. 1, 2026

(54) Packaging Solution Comprising Collagen Fragments

(71) Applicant: Bausch + Lomb Ireland Limited, Dublin (IE)

(72) Inventors: Jade J. Russell, Perry, NY (US); Alok K. Awasthi, Pittsford, NY (US); James Anthony DiBella, Penfield, NY (US)

(73) Assignee: Bausch + Lomb Ireland Limited, Dublin (IE)

(21) Appl. No.: 19/570,382

(22) Filed: Mar. 18, 2026

Related U.S. Application Data
(60) Provisional application No. 63/774,458, filed on Mar. 19, 2025.

(51) Int. Cl. A61L 12/14; A61L 12/08; A61L 101/46; G02C 7/04

(52) U.S. Cl. CPC A61L 12/14; A61L 12/086; G02C 7/049; A61L 2101/46

(57) ABSTRACT

The present invention relates to ophthalmic devices, in particular contact lenses, which are capable of gradually releasing one or more collagen fragments during wear. The present invention also provides methods for making ophthalmic devices of the invention and for using time-controlled release of one or more collagen fragments for improving comfort and/or treating eye conditions, such as dry eye disease.

### CROSS-REFERENCE TO RELATED APPLICATIONS

The present application claims the benefit of priority to U.S. Provisional Patent Application No. 63/774,458, filed Mar. 19, 2025, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 16, 2026, is named 01426-0009-00US and is 3,934 bytes in size.

### FIELD

The present invention relates to ophthalmic devices, in particular contact lenses, which are capable of gradually releasing one or more collagen fragments during wear. The present invention also provides methods for making ophthalmic devices of the invention and for using time-controlled release of one or more collagen fragments for improving comfort and/or treating eye conditions, such as dry eye disease.

### BACKGROUND

It is highly desirable that contact lens be as comfortable as possible for wearers. There is a decades long need to improve the comfort of the lenses. While many attempts have been made to address this problem, an estimated 50% of people who wear contact lenses still experience dryness or eye irritation throughout the day and particularly towards the end of the day. In fact, contact lens discomfort is recognized as the most common cause of contact lens wearer drop-out over the past four decades.

One of the widely used approaches to improve ocular comfort with contact lenses is to apply directly eye drops of an ocular lubricant into the wearer's eye while the lens is being worn, in order to provide some relief to some extent, e.g., the initial discomfort of wearers, discomfort suffering from dry-eye effects, or end-of-day discomfort. However, there are unavoidable disadvantages with this approach. For example, eye drops are typically applied only after a lens wearer is already suffering discomfort and as such do not prevent the discomfort from occurring. Furthermore, a user needs to easily and conveniently access eye drops to ease the discomfort and therefore must carry a bottle of eye drops with him/her. This adds cost and inconvenience to the lens wearers. Even more so, the high viscosity of such drops can lead to transient blurriness and/or reduced light transmissivity upon installation, leading to reduced vision and even safety concerns.

When a new contact lens is removed from its packaging, a small volume of the packaging solution remains on the lens as “carry over”. A packaging solution that provides increased carry over may improve the comfort of the lens. A packaging solution that also improves the lubricity and/or smoothness of the lens to touch would also be advantageous.

Therefore, there exists a need for hydrogel soft contact lenses which not only have initial insertion comfort but also are comfortable during the period of wear.

### SUMMARY OF THE INVENTION

The present invention relates to ophthalmic devices, in particular sterile contact lenses, immersed in a packaging solution and sealed in a container, wherein the packaging solution provides one or more advantages such as one or more advantages to the contact lens. An additional feature of the present invention is to provide a packaging solution for the storage of contact lenses, such as hydrogel (e.g. silicone hydrogel) contact lenses that can increase the insertion comfort of the contact lenses.

A further feature of the present invention is to provide a packaging solution for the storage of contact lenses that provides increased packaging solution carry over when the lens is removed from its container.

A further feature of the present invention is to provide a packaging solution for the storage of contact lenses that improves the lubricity and/or smoothness of the contact lens to the touch.

A further feature of the present invention is to provide a packaging solution that reduces the contact angle of a contact lens.

A further feature of the present invention is to provide a packaging solution that results in a lower contact angle of a contact lens, even after rinsing.

A further feature of the present invention is to provide a packaging solution for the storage of contact lenses that provides the above features without adversely affecting the optical clarity of the lens.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention, in part relates to an unworn sterile contact lens immersed in a packaging solution and sealed in a container, wherein the packaging solution has a viscosity of less than 15.0 cP and comprises one or more collagen fragments (as defined below).

The present invention relates to ophthalmic devices, in particular contact lenses, which gradually release one or more collagen fragments during wear. The present invention also provides methods for making ophthalmic devices of the invention and for using time-controlled release of one or more collagen fragments for improving comfort and/or treating eye conditions, such as dry eye disease.

The present invention, in another aspect, provides a process for making a soft contact lens comprising the steps of:
a) obtaining an uncured lens forming composition;
b) introducing an amount of the prepolymer composition in a mold for making a contact lens;
c) polymerizing the uncured lens forming composition in the mold to form the soft contact lens;
d) packaging the resultant soft contact lens in a container containing a packaging solution comprising one or more collagen fragments; and
e) sterilizing the soft contact lens in the package.

These and other aspects of the invention will become apparent from the following description of the presently preferred embodiments. The detailed description is merely illustrative of the invention and does not limit the scope of the invention, which is defined by the appended claims and equivalents thereof. As would be obvious to one skilled in the art, many variations and modifications of the invention may be affected without departing from the spirit and scope of the novel concepts of the disclosure.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings.

An “uncured lens forming composition” refers to a composition including reactive ingredients, and optionally non-reactive ingredients, used in forming contact lenses. The ingredients can include one or more hydrophilic monomers, oligomers, macromers, or polymers; and/or one or more hydrophobic monomers, oligomers, macromers, or polymers; and/or one or more silicone-containing monomers, oligomers, macromers, or polymers; and/or one or more crosslinkable prepolymers; or any combinations thereof. The uncured lens forming composition is capable of being crosslinked and/or polymerized thermally or actinically to form a hydrogel polymer matrix of a soft contact lens;

A “hydrogel” refers to a polymeric material which can absorb at least 10 percent by weight of water when it is fully hydrated. A hydrogel material can be obtained by polymerization or copolymerization of at least one hydrophilic monomer in the presence of or in the absence of additional monomers and/or macromers or by crosslinking of a prepolymer.

A “silicone hydrogel” refers to a hydrogel obtained by copolymerization of a polymerizable composition comprising at least one silicone-containing vinylic monomer or at least one silicone-containing macromer or a silicone-containing prepolymer.

“Hydrophilic,” as used herein, describes a material or portion thereof that will more readily associate with water than with lipids.

A “monomer” means a low molecular weight compound that can be polymerized actinically or thermally or chemically. Low molecular weight typically means average molecular weights less than 700 Daltons.

As used herein, “actinically” in reference to curing or polymerizing of a polymerizable composition or material or a lens-forming material means that the curing (e.g., crosslinked and/or polymerized) is performed by actinic irradiation, such as, for example, UV irradiation, ionized radiation (e.g. gamma ray or X-ray irradiation), microwave irradiation, and the like. Thermal curing or actinic curing methods are well-known to a person skilled in the art. Lens-forming materials are well known to a person skilled in the art.

A “vinylic monomer”, as used herein, refers to a low molecular weight compound that has an ethylenically unsaturated group and can be polymerized actinically or thermally. Low molecular weight typically means average molecular weights less than 700 Daltons.

The term “ethylenically unsaturated group” or “olefinically unsaturated group” is employed herein in a broad sense and is intended to encompass any groups containing at least one >C═C< group. Exemplary ethylenically unsaturated groups include without limitation acryloyl, methacryloyl, allyl, vinyl, styrenyl, or other C═C containing groups.

A “hydrophilic vinylic monomer”, as used herein, refers to a vinylic monomer which is capable of forming a homopolymer that can absorb at least 10 percent by weight water when fully hydrated. Suitable hydrophilic monomers are, without this being an exhaustive list, hydroxyl-substituted lower alkyl (C₁ to C₈) acrylates and methacrylates, acrylamide, methacrylamide, (lower allyl) acrylamides and -methacrylamides, ethoxylated acrylates and methacrylates, hydroxyl-substituted (lower alkyl) acrylamides and -methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, vinylically unsaturated carboxylic acids having a total of 3 to 5 carbon atoms, amino (lower alkyl)-(where the term “amino” also includes quaternary ammonium), mono(lower alkylamino) (lower alkyl) and di(lower alkylamino) (lower alkyl) acrylates and methacrylates, allyl alcohol and the like.

A “macromer” refers to a medium to high molecular weight compound or polymer that contains functional groups capable of undergoing further polymerizing/crosslinking reactions. Medium and high molecular weight typically means average molecular weights greater than 700 Daltons. Preferably, a macromer contains ethylenically unsaturated groups and can be polymerized actinically or thermally.

A “prepolymer” refers to a starting polymer which can be cured (e.g., crosslinked and/or polymerized) actinically or thermally or chemically to obtain a crosslinked and/or polymerized polymer having a molecular weight much higher than the starting polymer. A “crosslinkable prepolymer” refers to a starting polymer which can be crosslinked upon actinic radiation or heating to obtain a crosslinked polymer having a molecular weight much higher than the starting polymer.

The term “soluble”, in reference to a compound or material in a solvent, means that the compound or material can be dissolved in the solvent to give a solution with a concentration of at least about 0.05% by weight at room temperature (i.e., from about 22° C. to about 28° C.).

The term “insoluble”, in reference to a compound or material in a solvent, means that the compound or material can be dissolved in the solvent to give a solution with a concentration of less than 0.005% by weight at room temperature (as defined above).

As used in this application, the term “molecular weight” of a polymeric material (including monomeric or macromeric materials) refers to the number average molecular weight unless otherwise specifically noted or unless testing conditions indicate otherwise.

The present invention also provides a packaging system for the storage of contact lenses. These lenses can provide optical correction, wound care, drug delivery, diagnostic functionality or cosmetic enhancement or effect or a combination of these properties. The invention is applicable to soft, hydrogel contact lenses. As is understood by one skilled in the art, a lens is considered to be “soft” if it can be folded back upon itself without breaking. As used herein, a hydrogel contact lens refers to a polymeric lens that has the ability to absorb and retain water in an equilibrium state. In the context of the present description, a hydrogel lens can be a polymeric material that is free of a silicone-containing component, or a hydrogel lens can be a polymeric material that includes a silicone-containing component.

Many silicone-free hydrogel contact lenses are based on polymerizable lens formulations that include one or more monomers such as diacetone acrylamide (DA); N,N-dimethylacrylamide (DMA); 2-Hydroxyethyl methacrylate (HEMA); methacrylic acid (MAA); methyl methacrylate (MMA); N-carboxl vinyl ester (NCVE); N-vinyl pyrrolidone (NVP); glyceryl methacrylate (GMA); and 2-methacryloyloxyethyl phosphorylcholine (MPC). Some examples of hydrogel contact lens materials include materials having the following US Adopted Names (USANs): etafilcon, hilafilcon A, hilafilcon B, nelfilcon, nesofilcon, ocufilcon, and omafilcon.

Silicone-containing hydrogel contact lenses are frequently referred to as silicone hydrogel contact lenses. Many silicone hydrogel contact lenses are based on polymerizable lens formulations that include siloxane monomers, oligomers, or macromers. Some examples of silicone hydrogel contact lens materials include materials having the following USANs: aquafilcon, balafilcon, comfilcon, enfilcon, galyfilcon, kalafilcon, lenefilcon, lotrafilcon A, lotrafilcon B, samfilcon, and senofilcon.

The present contact lenses may be the polymerized reaction product of a polymerizable composition that comprises one or more hydrophilic monomers, one or more hydrophobic monomers, one or more silicone-containing monomers, oligomers, or macromers, one or more polymers, or combinations thereof in addition, the polymerizable compositions used to make the present lenses may include crosslinking agents, free radical initiators, tinting agents, UV absorbers, and the like. The present soft contact lenses may comprise, consist essentially of, or consist of, any of the foregoing contact lens materials identified by the USAN names above. Most preferably, the present soft contact lenses may comprise, consist essentially of, or consist of hilafilcon B, balafilcon, or kalafilcon.

The present contact lenses can be molded contact lenses, such as spin-cast molded or cast molded contact lenses, or lathed contact lenses. It can be appreciated that these types of contact lenses can have different physical features resulting from their method of manufacture. A cast molded contact lens refers to a contact lens obtained from a contact lens mold assembly formed from two contact lens mold sections in contact with each other to form a contact lens shaped cavity. In addition, a portion of the present contact lenses can be polished or smoothed after forming the contact lens. For example, a contact lens that has been cast molded or lathed, or both, can be polished to reduce transition areas or improve edge shapes to provide greater comfort compared to unpolished lenses.

The present contact lenses can be daily wear lenses or extended wear lenses. As used herein, an extended wear contact lens refers to a contact lens that is approved for wearing on a continuous basis for more than 24 hours. Each contact lens of the lens pair can be a daily disposable contact lens (i.e., a contact lens that is worn on a person's eye only once and then discarded). In comparison, as understood by persons of ordinary skill in the art, a daily wear lens is a lens that is worn on a person's eye, and is then cleaned and is worn on the person's eye for at least one additional time. It can be appreciated that daily disposable contact lenses can be physically different, chemically different, or both compared to daily wear and extended wear contact lenses. For example, formulations used to make daily wear or extended wear contact lenses are different than formulations used to make daily disposable contact lenses due to the economic and commercial factors in making substantially larger volumes of daily disposable contact lenses.

The present contact lenses are placed on a patient's eye such that the posterior surface of the lens faces the corneal epithelium of the eye of the patient.

When the contact lenses are cast molded contact lenses, the forming step comprises cast molding a polymerizable composition into the shape of a contact lens, separating the cast molded contact lens from a contact lens mold member, contacting the separated cast molded contact lens with a liquid, inspecting the separated cast molded contact lens, packaging the separated cast molded contact lens in a contact lens package, and/or sterilizing the contact lens in the package, or any combinations thereof.

The polymerizable composition is dispensed onto the concave surface of the first mold member. The second mold member is placed against the first mold member to form a contact lens mold assembly having a contact lens shaped cavity with the polymerizable composition located therein. The contact lens mold assembly is then exposed to heat or light to polymerize the polymerizable composition and form a polymerized contact lens product. The contact lens mold assembly is demolded by separating the first and second mold members. The polymerized contact lens product remains attached to the first or the second mold member and is then delensed or separated from the mold member. The delensed contact lens is contacted with a liquid, which may be a washing liquid, or it may be a packaging liquid. In some methods, the washing liquid includes one or more agents to help extract unreacted or partially reacted ingredients from the delensed contact lens product. Once the lenses are placed in a packaging liquid, the packages can be sealed, and sterilized.

The packaging solutions according to the present invention are physiologically compatible. Specifically, the solution must be “ophthalmically safe” for use with a lens such as a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations. The solution should be sterile in that the absence of microbial contaminants in the product prior to release must be statistically demonstrated to the degree necessary for such products. The liquid media useful in the present invention are selected to have no substantial detrimental effect on the lens being treated or cared for and to allow or even facilitate the present lens treatment or treatments. The liquid media are preferably aqueous-based. A particularly useful aqueous liquid medium is that derived from saline, for example, a conventional saline solution or a conventional buffered saline solution.

The pH of the present solutions should be maintained within the range of about 6.0 to about 8, and preferably about 6.5 to about 7.8. Suitable buffers may be added, such as: phosphate; borate; citrate; carbonate; tris-(hydroxymethyl)aminomethane (TRIS); bis(2-hydroxyethyl)-imino-tris-(hydroxymethyl)aminoalcohol (bis-tris); zwitterionic buffers such as N-[2-Hydroxy-1,1-bis(hydroxymethyl)ethyl]glycine (Tricine) and N-[2-Hydroxy-1,1-bis(hydroxymethyl)ethyl]glycine, MOPS; N-(Carbamoylmethyl) taurine (ACES); amino acids and amino acid derivatives; and mixtures thereof. Generally, buffers will be used in amounts ranging from about 0.05 to about 2.5 percent by weight, and preferably from about 0.1 to about 1.5 percent by weight of the solution.

If needed, the solutions of the present invention may be adjusted with tonicity agents, to approximate the osmotic pressure of normal lacrimal fluids, which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution, which will cause stinging, and eye irritation.

Examples of suitable tonicity adjusting agents include, but are not limited to, sodium and potassium chloride, dextrose, calcium and magnesium chloride and the like and mixtures thereof. These agents are typically used individually in amounts ranging from about 0.01 to about 2.5% w/v and preferably from about 0.2 to about 1.5% w/v. Preferably, the tonicity agent will be employed in an amount to provide a final osmotic value of at least about 200 mOsm/kg, preferably from about 200 to about 450 mOsm/kg, more preferably from about 250 to about 400 mOsm/kg, and most preferably from about 280 to about 370 mOsm/kg.

If desired, one or more additional components can be included in the packaging solution. Such additional component or components are chosen to impart or provide at least one beneficial or desired property to the packaging solution. Such additional components may be selected from components that are conventionally used in one or more ophthalmic device care compositions. Examples of such additional components include cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

Useful sequestering agents include, but are not limited to, disodium ethylene diamine tetraacetate, alkali metal hexametaphosphate, citric acid, sodium citrate and the like and mixtures thereof.

Useful antioxidants include, but are not limited to, sodium metabisulfite, sodium thiosulfate, N-acetylcysteine, butylated hydroxyanisole, butylated hydroxytoluene and the like and mixtures thereof.

The method of packaging and storing an ophthalmic lens according to the present invention includes at least packaging the ophthalmic lens immersed in the aqueous contact lens packaging solution described above. The method may include immersing the ophthalmic lens in an aqueous contact lens solution prior to delivery to the customer/wearer, directly following manufacture of the contact lens. Alternately, the packaging and storing in the solution of the present invention may occur at an intermediate point before delivery to the ultimate customer (wearer) but following manufacture and transportation of the lens in a dry state, wherein the dry lens is hydrated by immersing the lens in the contact lens packaging solution. Consequently, a package for delivery to a customer may include a sealed container containing one or more unused contact lenses immersed in an aqueous contact lens packaging solution according to the present invention.

### Suitable Collagens

Collagens suitable for use in the present invention include collagens derived from natural sources and those obtained from recombinant methods. Collagen suitable for use in this invention are those that exhibit sufficient solubility and stability in the ophthalmic solutions as described herein. Suitable recombinant collagens may be those that correspond to the naturally occurring human collagen. Additionally, modifications may be made to the naturally occurring sequences to improve their suitability for use within this invention. Most preferably, the recombinant collagen is a fragment of collagen that exhibits the desired properties.

As used herein, the term “recombinant collagen” refers to refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX, prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties). In some embodiments, the collagen described herein can be prepared using bovine Type I collagen. Collagens are characterized by a repeating triplet of amino acids, (Gly-X-Y) n, where X and Y may be any amino acid, preferably proline and 4-hydroxyproline. The term “collagen” is particularly preferably understood to mean a peptide having the repetitive motif (Gly-Pro-Y) n and/or (Gly-X-Hyp) m, where X and Y may be any amino acid. The structure of collagen may consist of three intertwined peptide chains of differing lengths.

The percentages cited in connection with the molecular weight distribution of the collagen peptides in the collagen peptide preparations according to the invention relate to % by weight in relation to all collagen peptides contained in the relevant collagen peptide preparation.

In connection with the present invention, the term “collagen peptide” can be understood to mean a peptide which has an amino acid sequence occurring in collagen as defined above. A “collagen peptide” may also be understood to mean a genetically modified collagen peptide, which was obtained by modifying the amino acid sequence of a naturally occurring collagen peptide,

In connection with the present invention, the term “recombinant DNA” denotes an artificially produced or manipulated DNA molecule, which has been produced in vitro by means of genetic engineering methods. In one embodiment, the recombinant DNA is composed of components from different organisms of origin.

In connection with the present invention, a “recombinant or recombinantly produced collagen peptide” is understood to mean a collagen peptide encoded by recombinant DNA.

One class of suitable recombinant collagen fragments is described in U.S. Pat. No. 11,673,940, incorporated herein in its entirety by reference. “Hydrolyzed collagen peptide preparation” as that term is used herein are the collagen peptides resulting from the hydrolysis of a recombinantly produced collagen peptide having a molecular weight in a range from 8 to 100 kDa, wherein the resulting collagen peptides have an average molecular weight of 1 to 7 kDa and a molecular weight in the range from 0.1 to 13.5 kDa.

Another class of suitable recombinant collagen is described in U.S. Pat. No. 12,134,640, incorporated herein in its entirety by reference. Such suitable collagens comprise a polypeptide comprising or consisting of an amino acid sequence derived from a collagen type VI, or a fragment, variant, fusion or derivative thereof, or a fusion of said fragment, variant of derivative thereof, wherein the polypeptide, fragment, variant, fusion or derivative is capable of killing or attenuating the growth of microorganisms.

It will be appreciated by persons skilled in the art that the collagen type VI may be from a human or non-human source. For example, the collagen type VI may be derived (directly or indirectly) from a non-human mammal, such as an ape (e.g. chimpanzee, bonobo, gorilla, gibbon and orangutan), monkey (e.g. macaque, baboon and colobus), rodent (e.g. mouse, rat) or ungulates (e.g. pig, horse and cow).

Thus, by “collagen type VI” (also “collagen VI”) we include naturally occurring human collagen type VI and homologues thereof, such as bovine collagen type VI. In one preferred embodiment, the polypeptide is derived from human collagen type VI.

By an amino acid sequence “derived from” collagen type VI we include amino acid sequences found within the amino acid sequence of a naturally occurring collagen type VI protein. In particular, we include amino acid sequences that comprise at least five contiguous amino acids from the sequence of a naturally occurring collagen type VI, but exclude the full length protein. For example, in one embodiment the amino acid sequence may contain at least 5 contiguous amino acids from collagen type VI, for example at least 6, 7, 8, 9, 10, 11, 12, 13, 14, 15, 16, 17, 18, 19, 20, 21, 22, 23, 24, 25, 30, 40, or 50 contiguous amino acids from collagen type VI. Thus, the amino acid sequence derived from collagen type VI corresponds to a fragment of collagen type VI.

In a further embodiment, the polypeptide may comprise or consist of an intact VWA domain.

By “VWA domain” we include the type A domains of von Willebrand factor, and domains showing homology to the type A domains of von Willebrand factor, as well as VWA-domain containing regions.

In one embodiment, the polypeptide is derived from the α3 chain of collagen type VI. Thus, the polypeptide may be derived from the α3N or α3C regions. For example, the polypeptide may be derived from the N2, N3 or C1 domain of the α3 chain of collagen type VI.

In an alternative embodiment, the polypeptide is derived from the α4 chain of collagen type VI.

In another alternative embodiment, the polypeptide is derived from the α5 chain of collagen type VI.

In a further alternative embodiment, the polypeptide is derived from the α6 chain of collagen type VI.

In a still further alternative embodiment, the polypeptide is derived from the α2 chain of collagen type VI, for example from the α2N region.

Yet another class of suitable recombinant collagen fragments is described in US Patent Application Publication US2025/0011392 A1, incorporated herein in its entirety by reference. Such collagen fragments can be derived from the amino acid sequences of Col1A1, Col1A2, and Col3A1. Non-limiting examples of such sequences are those described by Accession Nos. P02461.4 (human Col3A1) (www.ncbi.nlm.nih.gov/protein/124056490), NP 001029211.1 (bovine Col1A1) (www.ncbi.nlm.nih.gov/protein/77404252), NP 776945.1 (bovine Col1A2) (www.ncbi.nlm.nih.gov/protein/27806257) and NP 001070299.1 (bovine Col3A1) (www.ncbi.nlm.nih.gov/protein/116003881), which are incorporated herein by reference.

Such recombinant collagen fragments can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n).

In some embodiments, the collagen fragment disclosed herein can have a molecular weight from about 40 kDa to about 60 kDa. In some embodiments, the collagen fragment can have a molecular weight of about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, or about 60 kDa. In a particular embodiment, the collagen fragment can have a molecular weight of about 55 kDa.

In some embodiments, the collagen fragment described herein can have an amino acid sequence according to SEQ ID NO: 1. In some embodiments, the collagen fragment can have at least about 70%, at least about 75%, at least about 80%, about 85%, at least about 87.5%, at least about 90%, at least about 92.5%, at least about 95%, at least about 97.5%, at least about 98%, at least about 99% or 100% sequence identity, or similarity to SEQ ID NO: 1.

The amino acid sequence of SEQ ID NO: 1 is:

MYRNLIIATALTCGAYSAYVPSEPWSTLTPDASLESALKDYSQTFGIAIK
SLDADKIKRDSYDVKSGVAVGGLAGYPGPAGPPGPPGPPGTSGHPGSPGS
PGYQGPPGEPGQAGPSGPPGPPGAIGPSGPAGKDGESGRPGRPGERGLPG
PPGIKGPAGIPGFPGMKGHRGFDGRNGEKGETGAPGLKGENGLPGENGAP
GPMGPRGAPGERGRPGLPGAAGARGNDGARGSDGQPGPPGPPGTAGFPGS
PGAKGEVGPAGSPGSNGAPGQRGEPGPQGHAGAQGPPGPPGINGSPGGKG
EMGPAGIPGAPGLMGARGPPGPAGANGAPGLRGGAGEPGKNGAKGEPGPR
GERGEAGIPGVPGAKGEDGKDGSPGEPGANGLPGAAGERGAPGFRGPAGP
NGIPGEKGPAGERGAPGPAGPRGAAGEPGRDGVPGGPGMRGMPGSPGGPG
SDGKPGPPGSQGESGRPGPPGPSGPRGQPGVMGFPGPKGNDGAPGKNGER
GGPGGPGPQGPPGKNGETGPQGPPGPTGPGGDKGDTGPPGPQGLQGLPGT
GGPPGENGKPGEPGPKGDAGAPGAPGGKGDAGAPGERGPPAIAGIGGEKA
GGFAPYYG

The amino acid sequence of SEQ ID NO: 2 is:

DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG.

In some embodiments, the collagen fragment described herein can have an amino acid chain length from about 350 amino acids to about 600 amino acids and can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1. In some embodiments, the such a collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

b. Packaging Solutions Comprising Collagen Fragments

In some embodiments, the packaging solution can comprise from about 0.0005% to about 25% by weight of a recombinant collagen fragment, from about 0.001% to about 20% by weight of a recombinant collagen fragment, from about 0.01% to about 15% by weight of a recombinant collagen fragment, from about 0.1% to about 10% by weight of a recombinant collagen fragment, from about 0.5% to about 5% by weight of a recombinant collagen fragment, from about 0.7% to about 5% by weight of recombinant collagen fragment, from about 1% to about 5% by weight of recombinant collagen fragment, from about 2% to about 5% by weight of recombinant collagen fragment, from about 2% to about 5% by weight of recombinant collagen fragment, from about 3% to about 5% by weight of recombinant collagen fragment, from about 4% to about 5% by weight of recombinant collagen fragment, or about 5% by weight of recombinant collagen fragment.

In some embodiments, the packaging solution of the present invention can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a collagen fragment with a sequence according to SEQ ID NO: 1 or SEQ ID NO: 2. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of one or more hydrolysis products of a recombinant collagen fragment with sequences according to one or more of SEQ ID NOs: 1-2. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a mixture of a recombinant collagen fragment and one or more hydrolysis products of that recombinant collagen fragment. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a mixture of a recombinant collagen fragment with a sequence according to SEQ ID NO: 1 or SEQ ID NO: 2 and one or more hydrolysis products of that recombinant collagen fragment.

The pH of the present solutions should be maintained within the range of about 6.0 to about 8, and preferably about 6.5 to about 7.8. Suitable buffers may be added, such as: phosphate; borate; citrate; carbonate; tris-(hydroxymethyl)aminomethane (TRIS); bis(2-hydroxyethyl)-imino-tris-(hydroxymethyl)aminoalcohol (bis-tris); zwitterionic buffers such as N-[2-Hydroxy-1,1-bis(hydroxymethyl)ethyl]glycine (Tricine) and N-[2-Hydroxy-1,1-bis(hydroxymethyl)ethyl]glycine, MOPS; N-(Carbamoylmethyl) taurine (ACES); amino acids and amino acid derivatives; and mixtures thereof. Generally, buffers will be used in amounts ranging from about 0.05 to about 2.5 percent by weight, and preferably from about 0.1 to about 1.5 percent by weight of the solution.

If needed, the solutions of the present invention may be adjusted with tonicity agents, to approximate the osmotic pressure of normal lacrimal fluids, which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution, which will cause stinging, and eye irritation.

Examples of suitable tonicity adjusting agents include, but are not limited to, sodium and potassium chloride, dextrose, calcium and magnesium chloride and the like and mixtures thereof. These agents are typically used individually in amounts ranging from about 0.01 to about 2.5% w/v and preferably from about 0.2 to about 1.5% w/v. Preferably, the tonicity agent will be employed in an amount to provide a final osmotic value of at least about 200 mOsm/kg, preferably from about 200 to about 450 mOsm/kg, more preferably from about 250 to about 400 mOsm/kg, and most preferably from about 280 to about 370 mOsm/kg.

The method of packaging and storing an ophthalmic lens according to the present invention includes at least packaging the ophthalmic lens immersed in the aqueous contact lens packaging solution described above. The method may include immersing the ophthalmic lens in an aqueous contact lens solution prior to delivery to the customer/wearer, directly following manufacture of the contact lens. Alternately, the packaging and storing in the solution of the present invention may occur at an intermediate point before delivery to the ultimate customer (wearer) but following manufacture and transportation of the lens in a dry state, wherein the dry lens is hydrated by immersing the lens in the contact lens packaging solution. Consequently, a package for delivery to a customer may include a sealed container containing one or more unused contact lenses immersed in an aqueous contact lens packaging solution according to the present invention.

In one embodiment, the steps leading to the present ophthalmic device packaging system include (1) molding an ophthalmic device in a mold comprising at least a first and second mold portion, (2) removing the lens from the mold portions; (3) introducing the packaging solution of this invention and the ophthalmic lens into the container, and (4) sealing the container. Preferably, the method also includes the step of sterilizing the contents of the container. Sterilization may take place prior to, or most conveniently after, sealing of the container and may be effected by any suitable method known in the art, e.g., by balanced autoclaving of the sealed container at temperatures of about 120° C. or higher. Preferred packages are plastic blister packages, including a recess for receiving a contact lens and the package solution, where the recess is sealed with lidstock prior to sterilization of the package contents.

### Coefficient of Friction

Tribology is the study of how two surfaces interact with each other when in relative motion. One aspect of tribology that may be of importance to contact lenses is friction. Friction is a measure of a material's resistance to lateral motion when placed against a specific substrate. The relative friction between two surfaces may be described in terms of a coefficient of friction (COF), which is defined as the ratio of the lateral force (Fₓ) that is required to initiate and then sustain movement to the normal force (F_{N}). Further, there are two friction coefficients that may be considered, the peak (or static) and average (or kinetic). The static COF is a measure of how much Fₓ is needed to initiate relative motion of two surfaces and is typically the larger of the two values. Practically, for contact lenses, the static COF is related to the amount of force needed to start a blink cycle or for the lens to begin moving over the cornea. The kinetic COF is a measure of how much lateral force is needed to sustain movement at a particular velocity averaged over a finite period of time. This value is related to the amount of force required to sustain the blink over the course of the entire cycle and the ease of motion of the lens on the cornea (which may be further related to how much the lens moves on the cornea.

Various in vitro techniques are used to determine surface CoF in contact lenses however, an internationally agreed gold standard method does not currently exist. Such tribological testing can be performed, for example, on a TA Discovery Hybrid controlled stress rheometer equipped with a ring tool of ~50 nm surface roughness and designed to be used in an aqueous environment to eliminate effects of moisture loss from the surface. The tool is lowered onto the lens surface until a target normal applied force F_{N} 0.05N is reached. The static, low speed kinetic, and high speed kinetic frictional forces are measured in 3 succinct steps. Each value represents the average of 4-5 lenses. Controls can be run from the same lenses but do not contain recombinant collagen fragments nor subjected to a packaging solution containing recombinant collagen fragments.

To measure the static coefficient of friction the rub tool torque is ramped at 2 μN·m/s from 0 to 500 μN·m while monitoring the rub tool velocity (ν). When the torque applied to the rub tool exceeds the static friction force of the lens surface the tool begins to spin freely. The torque at the point the disk begins to spin, the radius of the disk, and the applied normal force are then utilized to calculate the static coefficient of friction.

To measure the Low Speed Kinetic Coefficient of Friction the rub tool is rotated at a constant ‘low’ speed of 0.25 rad/s and the tangential force recorded and averaged for 90 s to calculate the low speed kinetic CoF.

To measure the “High Speed Kinetic Coefficient of Friction” the rub tool rotation is increased to 40.0 rad/s for 30 s and again the tangential force is averaged and used to calculate the high speed kinetic CoF.

The packaged contact lens of the invention when removed from its container has a reduced static COF, a reduced low kinetic COF, and/or a reduced high kinetic COF compared to a contact lens of the same material packaged in a packaging solution that has no collagen fragments but is otherwise identical (i.e., a “control lens”). Preferably, the packaged contact lens of the invention has a mean static COF, a mean low kinetic COF, and/or a mean high kinetic COF upon removal from its container that is at least 10%, 20%, 25%, 30%, 40% or 50% lower than the corresponding mean COF of a control lens.

A lens removed from a packaging solution containing collagen fragments preferably exhibits a mean static COF of less than 0.4, or a mean low kinetic COF of less than 0.25, or a mean high kinetic COF of less than 0.4. The mean static COF is more preferably less than 0.25, less than 0.2, less than 0.15, or less than 0.1. The mean low kinetic COF is preferably less than 0.2, less than 0.15, less than 0.1, or less than 0.05. The mean high kinetic COF is preferably less than 0.35, less than 0.3, less than 0.25, or less than 0.20.

As used herein, an “unrinsed lens” is removed from its package with tweezers and gently shaken to remove excess packaging solution. The surface of an unrinsed lens to be measured is gently blotted with lens paper. On the other hand, a “rinsed lens” is removed from its package and placed in a hydration well with 4 mL PBS for 3 seconds then removed with tweezers and then blotted in the same manner.

An unrinsed contact lens of the invention preferably has a lower static COF, low kinetic COF, and/or high kinetic COF than a rinsed lens of the invention. Preferably, an unrinsed contact lens of the invention has a mean static COF, a mean low kinetic COF, and/or a mean high kinetic COF upon removal from its container that is at least 10%, 20%, 25%, 30%, 40% or 50% lower than the corresponding mean COF of a rinsed lens.

Wettability of lenses can be determined using a captive bubble contact angle technique using a First Ten Angstroms FTA-1000 drop Shape Instrument. Advancing and receding captive bubble contact angles were collected for each sample. The advancing contact angle is defined as the angle measured in water as the air bubble is retracting from the lens surface (water is advancing across the surface). All captive bubble data was collected using a high speed digital camera focused onto the sample/air bubble interface. The contact angle was calculated at the digital frame just prior to contact line movement across the sample/air bubble interface. The receding contact angle is defined as the angle measured in water as the air bubble is expanding across the sample surface (water is receding from the surface).

The packaged contact lens of the invention when removed from its container has a reduced captive bubble contact angle value compared to a control lens. Preferably, the packaged contact lens of the invention has a mean captive bubble contact angle value upon removal from its container that is at least 10%, 20%, 25%, 30%, 40% or 50% lower than the corresponding mean captive bubble contact angle value of a control lens.

A lens removed from a packaging solution containing collagen fragments preferably exhibits a captive bubble contact angle value of less than 80°. The mean captive bubble contact angle is more preferably less than 80°, less than 70°, less than 60°, less than 50°, or less than 40°.

An unrinsed contact lens of the invention preferably has a lower captive bubble contact angle value than a rinsed lens of the invention. Preferably, an unrinsed packaged contact lens of the invention has a mean captive bubble contact angle value upon removal from its container that is at least 10%, 20%, 25%, 30%, 40% or 50% lower than the corresponding mean captive bubble contact angle value of a rinsed lens.

Wettability of lenses can also be determined using a sessile drop technique measured using KRUSS DSA-100™ instrument at room temperature and using DI water as probe solution. Lenses are blotted dry with a kim wipe to remove surface water without drying the lens, and the lens is mounted on an orb-shaped mount. The cuvette is empty during testing, and the syringe is lowered from above and dispenses the water droplet onto the lens. Measurement is taken in the DSA 100-Drop Shape Analysis Software and recorded.

The packaged contact lens of the invention when removed from its container has a reduced sessile water contact angle value compared to a control lens. Preferably, the packaged contact lens of the invention has a mean sessile water contact angle value upon removal from its container that is at least 10%, 20%, 25%, 30%, 40% or 50% lower than the corresponding mean sessile water contact angle value of a control lens.

A lens removed from a packaging solution containing collagen fragments preferably exhibits a sessile water contact angle value of less than 80°. The mean sessile water contact angle is more preferably less than 80°, less than 70°, less than 60°, less than 50°, or less than 40°.

An unrinsed contact lens of the invention preferably has a lower sessile water contact angle value than a rinsed lens of the invention. Preferably, an unrinsed packaged contact lens of the invention has a mean sessile water contact angle value upon removal from its container that is at least 10%, 20%, 25%, 30%, 40% or 50% lower than the corresponding mean sessile water contact angle value of a rinsed lens.

The surface hydrophilicity of a contact lens (or a biomedical device or a material) is assessed by determining water-break-up time (WBUT), i.e., the time required for the water film to start breaking on the lens surface. Briefly, lenses are removed from the vial and placed in PBS (phosphate buffered saline) for at least two rinses of 30 minutes each and then transferred to fresh PBS in order to remove loosely bound packaging additives from the lens surface. Contact lenses are loaded onto a support pedestal which is submerged in an aqueous solution. A vertical lift stage brings the lens out of the solution, exposing the lens to air which is synchronized to a camera. A video is obtained of the lens dewetting and analyzed on frame by frame basis with a post-processing program in Matlab. Lenses exhibiting WBUT>10 seconds are considered to have a hydrophilic surface and are expected to exhibit adequate wettability (ability to support the tear film) on-eye.

The packaged contact lens of the invention when removed from its container has an increased WBUT value compared to a control lens. Preferably, the packaged contact lens of the invention has WBUT value upon removal from its container that is at least 10%, 20%, 25%, 30%, 40% or 50% greater than the corresponding mean WBUT value of a control lens.

A lens removed from a packaging solution containing collagen fragments preferably exhibits a WBUT of at least 10 seconds, preferably at least about 25 seconds, more preferably at least about 50 seconds, most preferably at least about 75 seconds.

An unrinsed contact lens of the invention preferably has a higher WBUT than a rinsed lens of the invention. Preferably, an unrinsed packaged contact lens of the invention has WBUT value upon removal from its container that is at least 10%, 20%, 25%, 30%, 40% or 50% greater than the corresponding mean WBUT value of a rinsed lens.

It is preferable that the presence of collagen fragments in the packaging solution not result in cloudiness or a significant reduction in spectral transmissivity in the contact lens contained therein. Accordingly, it is preferred that the spectral transmissivity of an unrinsed contact lens of the present invention be greater than 90%, more preferably greater than 92%, even more preferably greater than 94%, and most preferably greater than 96%, when measured by at least one of ISO 18369-3 or ANSI Z80.20.

1. A packaged contact lens comprising an unworn sterile contact lens immersed in a packaging solution and sealed in a container, wherein the packaging solution comprises one or more collagen fragments.

2. The packaged contact lens of claim 1, wherein said collagen fragments have an average molecular weight of 1 to 7 kDa and a molecular weight in the range from 0.1 to 13.5 kDa.

3. The packaged contact lens of claim 2, wherein the collagen fragment is from about 0.001% to about 20% by weight of the packaging solution.

4. The packaged contact lens of claim 1, wherein said collagen fragments are derived from the group of amino acid sequences consisting of collagen type VI, Col1A1, Col1A2, and Col3A1.

5. The packaged contact lens of claim 4, wherein the collagen fragment is from about 0.001% to about 20% by weight of the packaging solution.

6. The packaged contact lens of claim 5, wherein said collagen fragments are derived from collagen type VI.

7. The packaged contact lens of claim 6, wherein said collagen fragments are derived from the group of amino acid sequences consisting of the α2 chain of collagen type VI, the α3 chain of collagen type VI, the α4 chain of collagen type VI, the α5 chain of collagen type VI, and the α5 chain of collagen type VI.

8. The packaged contact lens of claim 7, wherein said collagen fragments comprise an intact VWA domain.

9. The packaged contact lens of claim 1, wherein said collagen fragment has the amino acid sequence of SEQ ID NO: 1.

10. The packaged contact lens of claim 9, wherein the collagen fragment is from about 0.001% to about 20% by weight of the packaging solution.

11. The packaged contact lens of claim 1, wherein said collagen fragment has an amino acid sequence with at least about 70% sequence identity to SEQ ID NO: 1.

12. The packaged contact lens of claim 11, wherein said collagen fragment has a molecular weight from about 40 kDa to about 60 kDa.

13. (canceled)

14. (canceled)

15. The packaged contact lens of claim 1, wherein said collagen fragment has the amino acid sequence of SEQ ID NO: 2.

16. The packaged contact lens of claim 1, wherein said collagen fragment has an amino acid sequence with at least about 70% sequence identity to SEQ ID NO: 2.

17. The packaged contact lens of claim 16, wherein said collagen fragment has an average molecular weight from about 40 kDa to about 60 kDa.

18. (canceled)

19. (canceled)

20. An ophthalmic product comprising a sealed package which comprises a packaging solution and a contact lens, wherein the packaging solution comprises a collagen fragment.

21. The ophthalmic product of claim 20, wherein said contact lens has a lower mean static COF when measured unrinsed than when measured rinsed.

22. The ophthalmic product of claim 20, wherein said contact lens has a lower mean low kinetic COF when measured unrinsed than when measured rinsed.

23. The ophthalmic product of claim 20, wherein said contact lens has a lower high kinetic COF when measured unrinsed than when measured rinsed.

24.-30. (canceled)
